(19) United States
(12) Patent Application Publication
(10) Pub. No.: US 2025/0009983 A1
(43) Pub. Date: Jan. 9, 2025

(54) INDEFLATION DEVICE AND INDEFLATION SYSTEM

(71) Applicant: TERUMO KABUSHIKI KAISHA, Tokyo (JP)

(72) Inventor: Takumi FUKUDA, Hadano-shi (JP)

(73) Assignee: TERUMO KABUSHIKI KAISHA, Tokyo (JP)

(21) Appl. No.: 18/889,543

(22) Filed: Sep. 19, 2024

Related U.S. Application Data
(63) Continuation of application No. PCT/JP2022/048461, filed on Dec. 28, 2022.

(30) Foreign Application Priority Data
Mar. 24, 2022 (JP) 2022-048884

(51) Int. Cl. A61M 5/32; A61M 5/315

(52) U.S. Cl. CPC A61M 5/3234; A61M 5/31576

(57) ABSTRACT

An indeflation device and an indeflation system capable of automating retraction of a bubble from an inside of a syringe of an indeflator and a tube connected to the syringe are provided. The indeflation device includes a holding portion that holds the indeflator connected to a medical catheter, a plunger driving unit that moves a plunger of the syringe of the indeflator held by the holding portion, a mechanism that inclines or vibrates the holding portion, and a control unit that controls driving of at least one of the plunger driving unit and the mechanism.

### CROSS-REFERENCES TO RELATED APPLICATIONS

This application is a continuation of International Application No. PCT/JP2022/048461 filed on Dec. 28, 2022, which claims priority to Japanese Application No. 2022-048884 filed on Mar. 24, 2022, the entire content of both of which is incorporated herein by reference.

### TECHNOLOGICAL FIELD

The present disclosure generally relates to an indeflation device and an indeflation system for retracting a bubble from an indeflator used to a medical catheter with liquid and an indeflation system.

### BACKGROUND DISCUSSION

A medical catheter is used for a diagnosis or treatment of a lesion area existing in a luminal organ such as a blood vessel or a vascular channel. The medical catheter includes a shaft including a lumen filled with a fluid such as a contrast agent or a normal saline solution.

In order to fill the shaft with the fluid, an indeflator that can grasp a pressure in the shaft is used. Prior to the diagnosis or medical treatment, a medical practitioner operates the indeflator while confirming the pressure and fills the fluid. Japanese Patent Application Publication No. 2015-181534 A discloses a priming device that includes a first syringe for decompression and a second syringe that fills a priming solution, and decompresses an inside of the catheter with the first syringe and connects to the second syringe so as to automatically fill the priming solution.

In a case where a medical practitioner operates an indeflator, and in a case where an automatic filling device disclosed in Japanese Patent Application Publication No. 2015-181534 A is used, existence of a bubble near a distal end in a shaft of a catheter to be inserted into a blood vessel should be avoided. For this purpose, in a case where the bubble exists in a syringe filled with a fluid in the indeflator or the automatic filling device and in a tube connected from the syringe to the shaft, it is necessary to move the bubble from the shaft toward the indeflator and avoid entry of the bubble into the shaft.

### SUMMARY

An indeflation device and an indeflation system are disclosed that are capable of automating retraction of a bubble from an inside of a syringe of an indeflator and a tube connected to the syringe.

An indeflation device according to the present disclosure includes a holding portion configured to hold an indeflator connected to a medical catheter, a plunger driving unit configured to move a plunger of a syringe of the indeflator held by the holding portion, a mechanism configured to incline or vibrate the holding portion, and a control unit configured to control driving of at least one of the plunger driving unit and the mechanism.

An indeflation system according to the present disclosure includes an indeflation device including a holding portion configured to hold an indeflator connected to a medical catheter, a plunger driving unit configured to move a plunger of a syringe of the indeflator held by the holding portion, and a mechanism configured to incline or vibrate the holding portion and a control device configured to determine which one of the plunger driving unit and an inclination or a vibration of the mechanism is driven and to instruct the indeflation device on determined control content.

A method for retracting a bubble from an indeflation system according to the present disclosure includes holding an indeflator connected to a medical catheter in a recess of a casing of the indeflation system; and one or more of moving a plunger of a syringe of the indeflator held in the recess of the inflation system, the syringe of the indeflator being held in the recess of the inflation system, and inclining or vibrating the recess of the casing of the indeflation system.

### BRIEF DESCRIPTION OF THE DRAWINGS

FIG. 1 is a schematic view of a medical system including an indeflation device.

FIG. 2 is a schematic perspective view of the indeflation device.

FIG. 3 is a schematic side view of the indeflation device.

FIG. 4 is a block diagram illustrating a configuration of the indeflation device.

FIG. 5 is a flowchart illustrating an example of a processing procedure by a processing unit of the indeflation device.

FIG. 6 is a flowchart illustrating an example of the processing procedure by the processing unit of the indeflation device.

FIG. 7 is a flowchart illustrating an example of the processing procedure by the processing unit of the indeflation device.

FIG. 8 is a schematic perspective view of an indeflator and an indeflation device according to a second embodiment.

FIG. 9 is a block diagram illustrating a configuration of the indeflation device according to the second embodiment.

FIG. 10A is a flowchart illustrating an example of a processing procedure by a processing unit in the second embodiment.

FIG. 10B is a flowchart illustrating an example of the processing procedure by the processing unit in the second embodiment.

FIG. 11 is a schematic view of an indeflation system according to a third embodiment.

FIG. 12 is a block diagram illustrating a configuration of an indeflation device according to the third embodiment.

FIG. 13 is a block diagram illustrating a configuration of a control device.

FIG. 14A is a flowchart illustrating an example of a processing procedure of the indeflation system according to the third embodiment.

FIG. 14B is a flowchart illustrating an example of the processing procedure of the indeflation system according to the third embodiment.

FIG. 15 is a schematic perspective view of an indeflation device according a fourth embodiment.

FIG. 16 is a block diagram illustrating a configuration of the indeflation device according to the fourth embodiment.

### DETAILED DESCRIPTION

Set forth below with reference to the accompanying drawings is a detailed description of embodiments of an indeflation device and an indeflation system for retracting a bubble from an indeflator used to a medical catheter with liquid and an indeflation system.

### First Embodiment

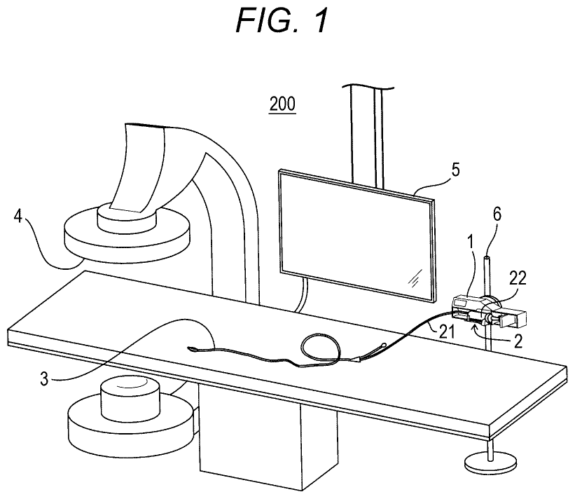

FIG. 1 is a schematic view of a medical system 200 including an indeflation device 1. The indeflation device 1 is a device that controls supply of a fluid from an indeflator 2 connected to a catheter 3 to be inserted into a luminal organ of a patient to the catheter 3. The medical system 200 further includes an imaging device 4 that images a medical image of the luminal organ of the patient into which the catheter 3 is inserted and a display apparatus 5 that monitor-outputs the medical image.

The imaging device 4 is a device that monitor-outputs the medical image of the luminal organ in which the catheter is inserted. First, the medical image, such as an angiographic image, is an image obtained by emitting X-rays. The medical image may be an image imaged by another method, as long as a state of the luminal organ can be observed during the catheter 3 is inserted, in addition to the angiographic image. Data of the medical image imaged by the imaging device 4 is displayed on the display apparatus 5 in real time.

The catheter 3 is a medical flexible tube. In particular, a balloon is attached to a distal end of the catheter 3. The balloon can be made of, for example, resin and is more flexible than a shaft of the catheter 3. A lumen in the shaft of the catheter 3 communicates with the balloon, and the balloon and the lumen are filled with a fluid such as a contrast agent provided from the indeflator 2 connected to the lumen. The balloon is inflated by pressurizing the fluid in the balloon and the lumen with the indeflator 2, and the balloon is deflatable to an original size by depressurizing the fluid.

The indeflator 2 is an instrument that is connected to the catheter 3 and supplies the fluid such as a contrast agent or a normal saline solution to the catheter 3. The indeflator 2 includes a tube 21 connected to the catheter 3 and a syringe 22 that is connected to the tube 21 and is filled with the fluid.

An operator who uses the medical system 200 or an assistant causes the indeflation device 1 to hold the indeflator 2, before connecting the indeflator 2 to the catheter 3 and performing an operation. The indeflation device 1 has a function for automatically supplying the fluid from the holding indeflator 2 to the catheter 3. The indeflation device 1 has a function for performing control to prevent bubbles from flowing into the catheter 3, in a case where the bubbles exist in a portion of the indeflator 2 filled with the fluid at the time of automatic supply. Hitherto, it has been necessary to prepare for removal of air bubbles before the operator uses the indeflator 2. However, by using the indeflation device 1 that can automatically avoid a risk of the air bubbles being mixed into the catheter 3, it is possible to reduce a workload of the operator or the assistant.

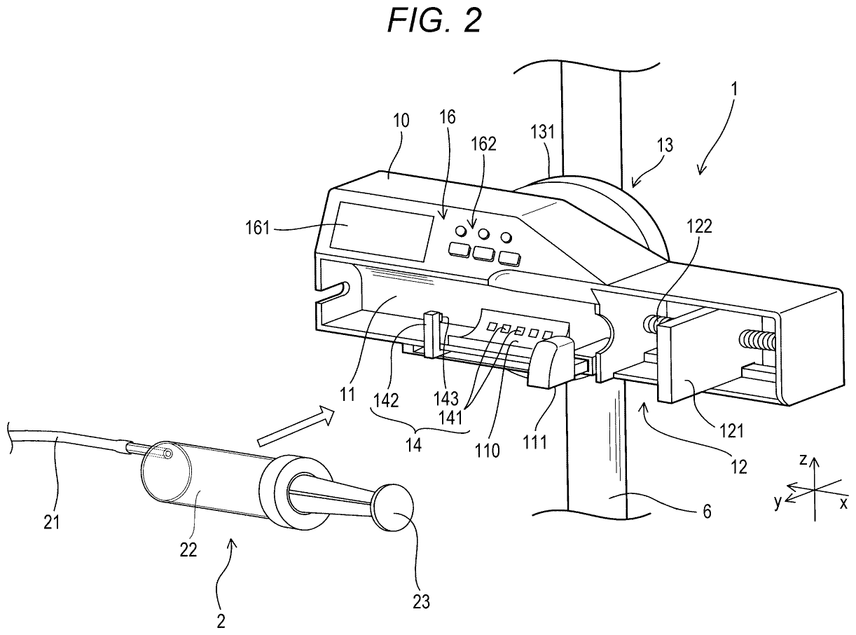

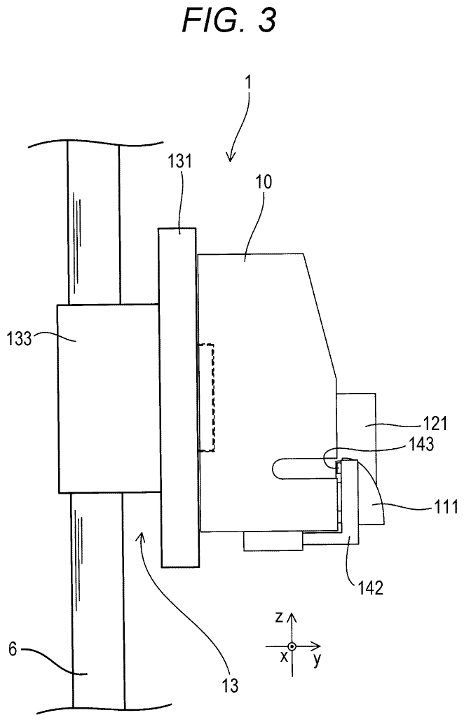

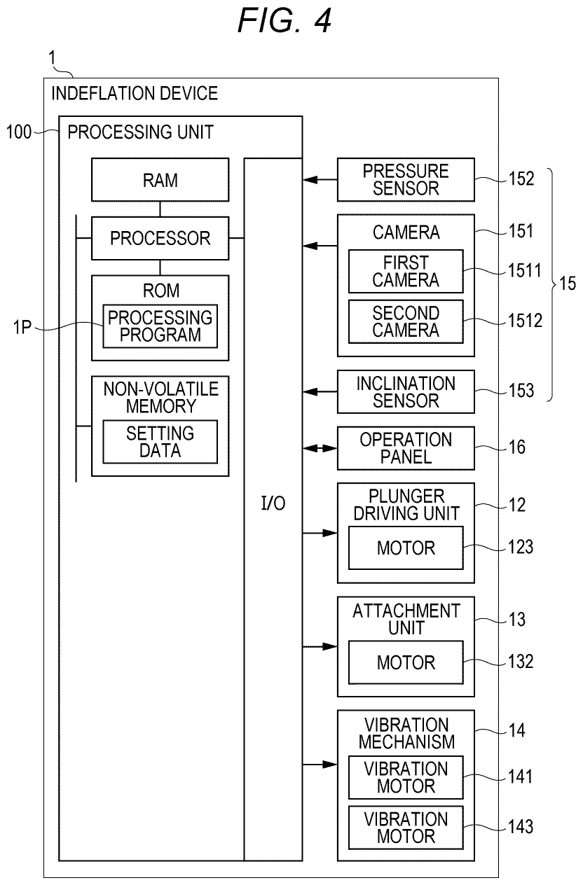

Details of the indeflation device 1 will be described. FIG. 2 is a schematic perspective view of the indeflation device 1, FIG. 3 is a schematic side view of the indeflation device 1, and FIG. 4 is a block diagram illustrating a configuration of the indeflation device 1. In the following description, an x direction corresponding to a length direction of the held indeflator 2, a y direction corresponding to a depth direction of the device, and a z direction corresponding to the vertical direction, indicated by arrows in FIGS. 2 and 3, are used.

As illustrated in FIGS. 1 and 2, the indeflation device 1 includes a casing 10 having an elongated shape and holds the indeflator 2 in a recess (holding portion 11) provided along a long side direction. The indeflation device 1 is used by being attached to a support column 6 such that the length direction of the held indeflator 2 is along the substantially horizontal direction.

The indeflation device 1 includes the holding portion 11 that holds the syringe 22 included in the indeflator 2, a plunger driving unit 12 that moves a plunger 23 of the syringe 22, an attachment unit 13 to the support column 6, a vibration mechanism 14, a sensor group 15 used to specify a state of the indeflator 2, an operation panel 16, and a processing unit 100 that executes processing for controlling each unit.

The holding portion 11 has a holding surface 110 having a semicircular cross section with a length direction of the casing 10 as an axis and a clamp 111 used to fix the syringe 22. The holding surface 110 holds an outer cylinder side ‘surface of the syringe 22 of the indeflator 2. The clamp 111 is slidable in a direction across the holding surface 110 so as to hold the indeflators 2 having a plurality of sizes. The clamp 111 holds and fixes the syringe 22 in a width direction and locks a flange on a proximal end side and fixes the length direction of the syringe 22. In a portion of the clamp 111 in contact with the syringe 22, a cushioning material 112, for example, made of a soft material such as rubber or felt is provided.

The plunger driving unit 12 includes a slider 121 that has a surface in contact with an end surface of the plunger 23 of the indeflator 2 held by the holding portion 11, a feed screw 122 provided in parallel to an axial direction of the held indeflator 2, and a motor 123 that rotates the feed screw 122. The slider 121 is integrated with a nut of the feed screw 122 and moves the slider 121 in the axial direction of the syringe 22 along with rotation of the feed screw 122. The motor 123 rotates the feed screw 122 in one of bidirectional directions at a speed on which an instruction is given, in response to a signal from the processing unit 100. A fixing tool used to fix the plunger 23 is provided on a contact surface of the slider 121 in contact with the plunger 23. The flange of the plunger 23 is fixed to the slider 121 with the fixing tool, and the plunger 23 and the slider 121 can be integrated. As a result, the plunger 23 can be bidirectionally moved by rotating the motor 123, and both of pressurization and depressurization of the syringe 22 can be realized. The plunger driving unit 12 can recognize a movement amount from a reference position of the nut, that is, the plunger 23 (one end of movable range), on the basis of a rotation amount of the feed screw 122.

A configuration of the plunger driving unit 12 is not limited to a configuration that realizes push and pull as described above with reference to FIG. 2. For example, the plunger 23 may be a screw type with respect to the syringe 22, and an inner cylinder of the syringe 22 may be moved by rotating a proximal end of the plunger 23 with a motor. In this case, in the plunger driving unit 12, it is sufficient to provide the motor that rotates the proximal end of the plunger 23 and a transmission unit for the rotation of the motor.

The attachment unit 13 includes a plate 131 rotatably provided along a rear surface with a normal line of the rear surface as an axis and a motor 132 that rotates the plate 131, on the rear surface of the casing 10. On a surface of the plate 131 opposite to the casing 10, a clip-like fixing tool 133 for fixing to the support column 6 is provided. The fixing tool 133 is provided so that the length direction of the casing 10 is fixed to the support column 6 to be rotated along a vertical surface while maintaining the rear surface in the vertical direction. The casing 10 that holds the indeflator 2 with respect to the plate 131 fixed to the support column 6 can rotate around the axis orthogonal to the support column 6 and the length direction of the casing 10, depending on a rotation amount of the motor 132. The attachment unit 13 functions as an inclination mechanism that makes the indeflator 2 incline in the length direction. The attachment unit 13 is connected to the processing unit 100, and a rotation direction and a rotation amount of the motor is controlled by the processing unit 100.

The attachment unit 13 may include a hemispherical recess provided in the rear surface of the casing 10, a spherical protrusion fitted into the recess, a gear that rotates the recess with respect to the protrusion around each of two orthogonal directions as axes, and a motor that rotates the gear. As a result, the attachment unit 13 can function as an inclination mechanism that rotates the indeflator 2 not only in the length direction but also in a direction substantially parallel to the length direction, that is, makes the indeflator 2 incline in a vertical direction.

The vibration mechanism 14 includes a plurality of vibration motors 141 provided along the length direction, on the holding surface 110 of the holding portion 11. The vibration mechanism 14 includes a vibration motor 143 that is attached to a slider 142 movable in the length direction with respect to the casing 10 and has contact with the outer cylinder side surface of the syringe 22. It is sufficient for the vibration mechanism 14 to include at least one of the vibration motor 141 provided on the holding surface 110 and the vibration motor 143 attached to the slider 142. Intensity of vibrations of the vibration motors 141 and 143 can be controlled by the processing unit 100.

The sensor group 15 includes a camera 151 of which an imaging range is the syringe 22 and the tube 21, a pressure sensor 152 that estimates a pressure in the syringe 22, and an inclination sensor 153 that detects the inclination of the indeflator 2, that is, the syringe 22.

The camera 151 is provided above the holding surface 110 and faces downward towards the holding surface. The camera 151 includes a first camera 1511 that images a nozzle portion of the syringe 22 from an end surface side in an oblique direction and a second camera 1512 that images the tube 21 connected to the nozzle from above. The first camera 1511 and the second camera 1512 may further include a plurality of cameras, and in addition, an installation position and an imaging direction are not limited to this. The camera 151 outputs an image signal of the imaged image to the processing unit 100.

The pressure sensor 152 is provided on a contact surface of the plunger 23 with the slider 121 of the plunger driving unit 12. The pressure sensor 152 measures a reaction force from the plunger 23 in a case where the slider 121 is moved to pressurize the syringe 22 and calculates a pressure from dimensions of the syringe 22. The pressure sensor 152 can be, for example, a load cell, a strain sensor, or the like. The pressure sensor 152 outputs a signal corresponding to the measured reaction force to the processing unit 100. The pressure sensor 152 may also measure a pressure in the syringe 22. The processing unit 100 may receive and use a measurement result output from the pressure sensor 152 provided in the indeflator 2.

The inclination sensor 153 is provided to be parallel to the length direction of the holding portion 11, that is, the held indeflator 2, in a part of the casing 10. The inclination sensor 153 detects an angle from the horizontal direction and outputs the angle to the processing unit 100. The processing unit 100 can detect an inclination of the held indeflator 2 from the horizontal direction, according to the output from the inclination sensor 153.

The operation panel 16 includes a display 161 that displays the state of the indeflator 2 and a physical button 162 that receives an operation. The physical buttons 162 can be a start button and a stop button. Note that the operation panel 16 may include not only the physical buttons 162 but also a touch panel built in the display 161 and may receive an operation with the touch panel, instead of the physical button 162.

The processing unit 100 is fixed in the casing 10, and is connected to the plunger driving unit 12, the motor of the attachment unit 13, the vibration motors 141 and 143 of the vibration mechanism 14, the sensor group 15, and the operation panel 16 with signal lines.

The processing unit 100 includes a processor such as a central processing unit (CPU) or a micro-processing unit (MPU) and a memory such as a read only memory (ROM) or a random access memory (RAM). The processing unit 100 can be, for example, a microcontroller. The ROM stores a processing program 1P, setting data, or the like. The processor takes the image signal obtained from the camera 151, in response to the operation received by the operation panel 16, on the basis of this processing program 1P, and executes control processing on any one or the plurality of the plunger driving unit 12, the attachment unit 13 that functions as the inclination mechanism, and the vibration mechanism 14, on the basis of the image. The setting data is data for bubble detection used in processing to be described later and data of a first size, a second size, or the like.

In the indeflation device 1 according to the first embodiment configured in this way, the indeflation device 1 itself is configured to be inclined by the attachment unit 13 so as to move the bubbles toward the plunger 23, and the indeflation device 1 can be inclined so as to raise the plunger 23 side of the indeflator 2. Furthermore, the indeflation device 1 is configured to be able to vibrate the vibration mechanism 14 to apply stimulation to the bubbles in the fluid. Moreover, the indeflation device 1 is configured to be able to draw the plunger 23 by the plunger driving unit 12 in order to move the bubbles and draw the bubbles from the tube 21 into the syringe 22.

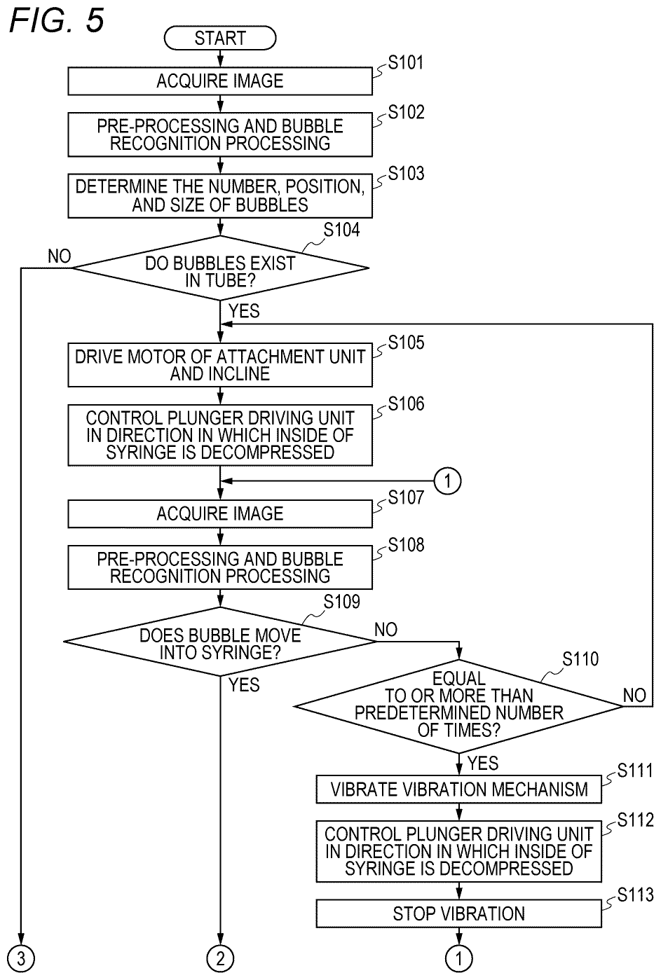

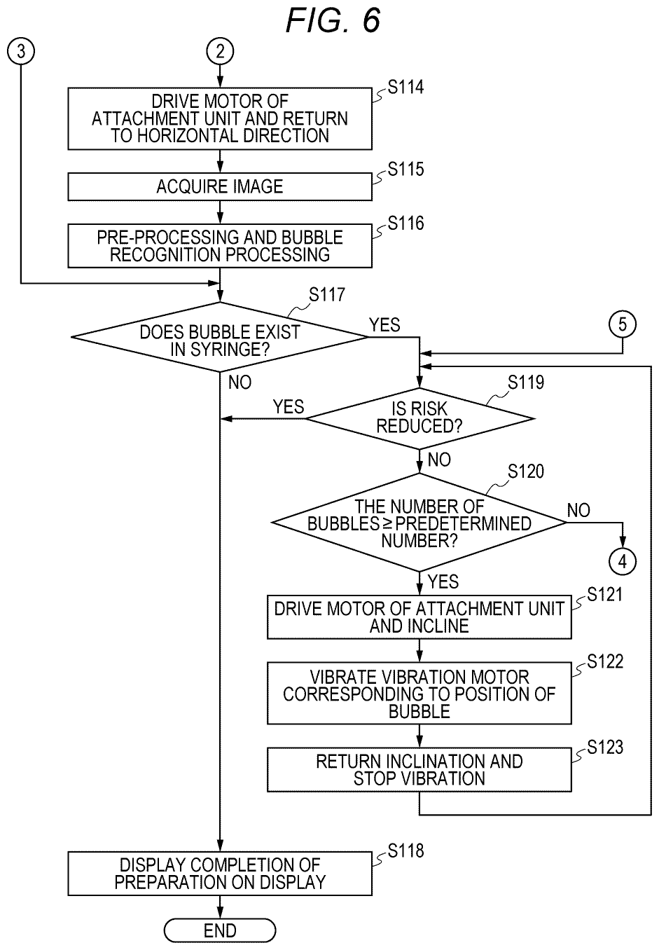

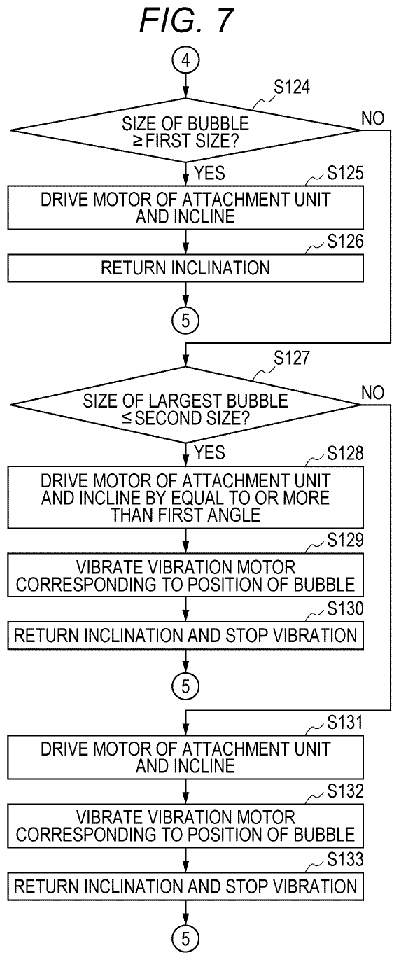

Processing for controlling the indeflation device 1 configured in this way and avoiding the movement of the bubbles to the catheter 3 will be described. FIGS. 5 to 7 are flowcharts illustrating an example of a processing procedure by the processing unit 100 of the indeflation device 1. When an operator such as an operator or an assistant houses the indeflator 2 filled with the fluid such as the contrast agent in the holding portion 11 of the indeflation device 1, confirms that the syringe 22 is fixed with the clamp 111, and turns on power, the following processing is executed.

Before starting automatic pressing processing, the processing unit 100 of the indeflation device 1 executes the following processing to avoid a risk that the bubbles move toward the catheter 3.

The processing unit 100 acquires an image from the camera 151 for confirmation (S101) and executes pre-processing such as noise removal or edge processing on the acquired image and executes bubble recognition processing for each image (S102).

In S101, the processing unit 100 can capture (acquire) an image at any timing from the image signal monitor-output from the first camera 1511 that images the syringe 22 and capture (acquire) an image at any timing from the image signal output from the second camera 1512 that images the tube 21.

In S102, the processing unit 100 may determine whether the image matches a pattern of the bubbles or may recognize an image by an image recognition model learned to output a range of the bubbles in a case where the image is input. In S102, in a case where the bubbles are imaged in the image, the processing unit 100 obtains coordinate information in the image of the bubble range in the image.

The processing unit 100 determines a result of the recognition processing, the number of bubbles, and a position and a size of each bubble (S103). In a case where the bubbles are not recognized, the processing unit 100 determines in S103 that the number of bubbles is zero. In a case where it is recognized that a bubble (or bubbles) is imaged in the image acquired from the first camera 1511 that images the syringe 22, the processing unit 100 determines that the position of the bubble is in the syringe 22. In a case where it is recognized that a bubble (or bubbles) is imaged in the image acquired from the second camera 1512 that images the tube 21, the processing unit 100 determines that the position of the bubble is in the tube 21. The processing unit 100 may store correspondence between coordinates in the image in association with a position and an angle of view of the first camera 1511 and a position in the syringe 22 and distinguish the position of the bubble in detail as a distal end (nozzle) side in the syringe 22, a central portion, a proximal end side, a top surface side and a bottom surface side in the syringe 22, or a combination of the distal end (nozzle) side in the syringe 22, the central portion, the proximal end side, the top surface side, and the bottom surface side in the syringe 22.

In S103, in a case where a plurality of bubbles exists in the image, the processing unit 100 allocates identification data to each bubble, and determines a size and a position in association with each piece of the identification data. The processing unit 100 may allocate the identification data as a cluster in a case where the plurality of bubbles is close to each other in S103.

As a result of the processing in S103, the processing unit 100 determines whether or not the bubbles exist in the tube (S104). In a case where it is determined that the no bubble exists in the tube (S104: NO), the processing unit 100 proceeds the processing to next S116.

In a case where it is determined that the bubble exists in the tube 21 (S104: YES), the processing unit 100 inclines the indeflation device 1 at a predetermined angle by driving the motor 132 of the attachment unit 13 (S105) and controls the plunger driving unit 12 so as to move the plunger 23 in a direction in which the inside of the syringe 22 is decompressed for a predetermined time (for example, five seconds) (S106). After a predetermined time has elapsed, the processing unit 100 moves the plunger 23 to a position before the decompression.

The processing unit 100 acquires an image after the predetermined time has elapsed (S107), executes the pre-processing and the recognition processing on the acquired image (S108), and determines whether or not the bubble in the tube 21 has moved into the syringe 22 (S109). In a case where it is determined that the bubble has not moved into the syringe 22 (S109: NO), it is determined whether or not the number of times that the plunger 23 has been pulled is equal to or more than a predetermined number of times (S110). In a case where it is determined in S110 that the number of times that the plunger 23 has been pulled is not equal to or more than the predetermined number of times (S111: NO), the processing unit 100 returns the processing to S105.

In a case where the bubble does not move even if the processing for making an inclination by the attachment unit 13 and performing decompression by pulling the plunger 23 the predetermined number of times, that is, in a case where it is determined in S110 that the number of times that the plunger 23 has been pulled is equal to or more than the predetermined number of times (S110: YES), the processing unit 100 vibrates the vibration motor 141 at a position closest to the nozzle of the syringe 22 (S111). In S111, the processing unit 100 may vibrate the vibration motor 143 by moving the slider 142 to the position closest to the nozzle of the syringe 22. The processing unit 100 controls the plunger driving unit 12 so as to move the plunger 23 to decompress the inside of the syringe 22 (S112). At this time, the indeflation device 1 itself remains to be inclined. After the predetermined time has elapsed, the processing unit 100 returns the position of the plunger 23 to the position before the decompression, stops the vibration (S113), and returns the processing to S107. However, in a case where it is determined that the bubble in the tube 21 does not move into the syringe 22, a message providing a notification that the bubble does not move may be displayed on the display 161 of the operation panel 16, or buzzer sound may be output by providing a sound output unit.

In a case where it is determined that the bubble in the tube 21 has moved into the syringe 22 (S109: YES), the processing unit 100 returns the inclination of the indeflation device 1 itself, that is, the indeflator 2 to the horizontal direction (S114).

The processing unit 100 acquires the image from the camera 151 (S115), executes the pre-processing and the recognition processing (S116), and determines whether or not the bubble exists in the syringe 22 (S117).

In a case where it is determined that the bubble does not exist in the tube 21 and the syringe 22 from the beginning (S117: NO), the processing unit 100 displays completion of preparation of the indeflator 2 on the operation panel 16 (S118), and ends the processing. Since the preparation is completed, the processing unit 100 can execute, for example, processing for inflating the balloon provided at the distal end of the catheter 3, supplying the normal saline solution, or the like as soon as pressing of the start button is detected.

The determination that the bubble does not exist in the tube 21 and the syringe 22 after once it is determined in S117 that the bubble exists in the tube 21 is based on that an air reservoir, an exhaust port, or the like used to remove the bubble is provided in the syringe 22 or the tube 21 via a valve (refer to second embodiment). In this case, the processing unit 100 controls the inclination by the motor 132 of the attachment unit 13, the vibration by the vibration motors 141 and 143 of the vibration mechanism 14, and the decompressing and the pressurization by the plunger driving unit 12, so as to move the detected bubble to a position the valve to the air reservoir or the exhaust port.

In a case where it is determined in S117 that the bubble exists in the syringe (S117: YES), the processing unit 100 determines whether or not the risk such that the bubble moves from the tube 21 to the catheter 3 is sufficiently reduced, on the basis of the number, the size, and the position of the bubbles in the syringe 22 (S119).

In S119, the processing unit 100 can recognize the movement of the bubble across continuous images, by continuously executing image processing on the images of the image signal monitor-output.

In S119, in a case where the bubbles exist in the syringe 22 and the detected bubbles are collected to bubbles of a predetermined number or less, are sufficiently large, and are positioned on the side of the plunger 23, the processing unit 100 determines that the risk is sufficiently reduced.

In a case where it is determined that the risk is sufficiently reduced (S119: YES), the processing unit 100 proceeds the processing to S118.

In a case where it is determined that the risk is not sufficiently reduced (S119: NO), the processing unit 100 determines whether or not the number of bubbles is equal to or more than the predetermined number (S120). In a case where it is determined that the number of bubbles is equal to or more than the predetermined number (S120: YES), the processing unit 100 inclines the indeflation device 1 at a predetermined angle by the attachment unit 13, so as to collect and move the bubbles (S121) and vibrates the vibration motor corresponding to the position of the bubble (S122). In S121 and S122, the processing unit 100 may control the plunger driving unit 12 so as to decompress (or pressurize) the inside of the syringe 22. The processing unit 100 returns the inclination, stops the vibration mechanism 14 (S123) and returns the processing to S119.

In a case where it is determined that the number of bubbles is less than the predetermined number (S120: NO), the processing unit 100 determines whether or not the size of the bubble in the syringe 22 is equal to or larger than a predetermined first size (S124). The first size can be, for example, a size of a relatively large bubble such as five millimeters. The first size may be determined in advance based on not only an actual side but also the number of pixels.

In a case where it is determined that the size of the largest bubble is equal to or larger than the predetermined first size (S124: YES), the processing unit 100 makes inclination equal to or larger than a predetermined first angle by the attachment unit 13 (S125), returns the inclination to the horizontal direction after a predetermined time of standby (S126), and returns the processing to S119. The sufficiently large bubble easily moves to the proximal end side of the syringe 22 only by the inclination. The predetermined first angle can be a relatively large angle, for example, 30 degrees.

In a case where it is determined in S124 that the size of the largest bubble is less than the predetermined first size (S124: NO), the processing unit 100 determines whether or not the size of the largest bubble is equal to or less than a predetermined second size (S127). The second size is smaller than the predetermined first size and can be a size of a relatively small bubble, for example, two millimeters. The second size may be determined based on not only the actual size but also the number of pixels.

In a case where it is determined that the size of the largest bubble is equal to or less than the predetermined second size (S127: YES), it cannot be expected that the bubbles move only by the inclination. Therefore, the processing unit 100 inclines the attachment unit 13 by the predetermined first angle or more (S128) and vibrates the vibration motors 141 and 143 corresponding to the position of the bubble (S129). In S128, the processing unit 100 moves the vibration motor 143 to the position of the bubble with the slider 142, and then, vibrates the vibration motor 143. The processing unit 100 may control the plunger driving unit 12 to perform decompression. The processing unit 100 returns the inclination to the horizontal direction after the predetermined time of standby and stops the vibration motor (S130) and returns the processing to S119.

In a case where it is determined in S127 that the size of the largest bubble is larger than the predetermined second size (S127: NO), the number of bubbles is less than the predetermined number, and the size of the bubble is less than the first size and larger than the second size. In this case, the processing unit 100 inclines the indeflation device 1 with the attachment unit 13 (S131) and vibrates the vibration motors 141 and 143 corresponding to the position of the bubble (S132). The processing unit 100 returns the inclination to the horizontal direction after the predetermined time of standby and stops the vibration motor (S133) and returns the processing to S119.

In this way, the indeflation device 1 can avoid a risk that the bubble is mixed into the side of the catheter 3, using at least one of the vibration mechanism 14, the attachment unit 13 that is the inclination mechanism, and the plunger driving unit 12.

### Second Embodiment

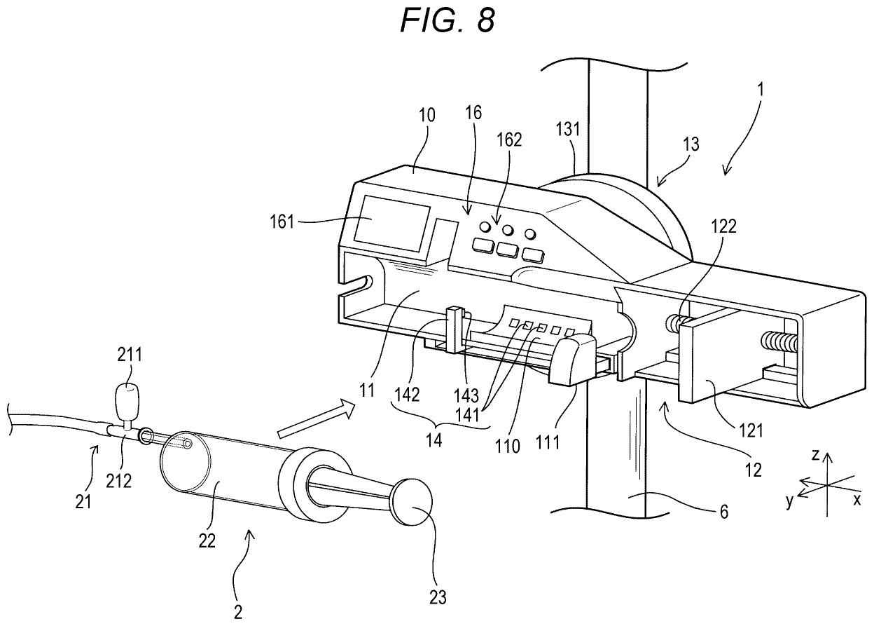

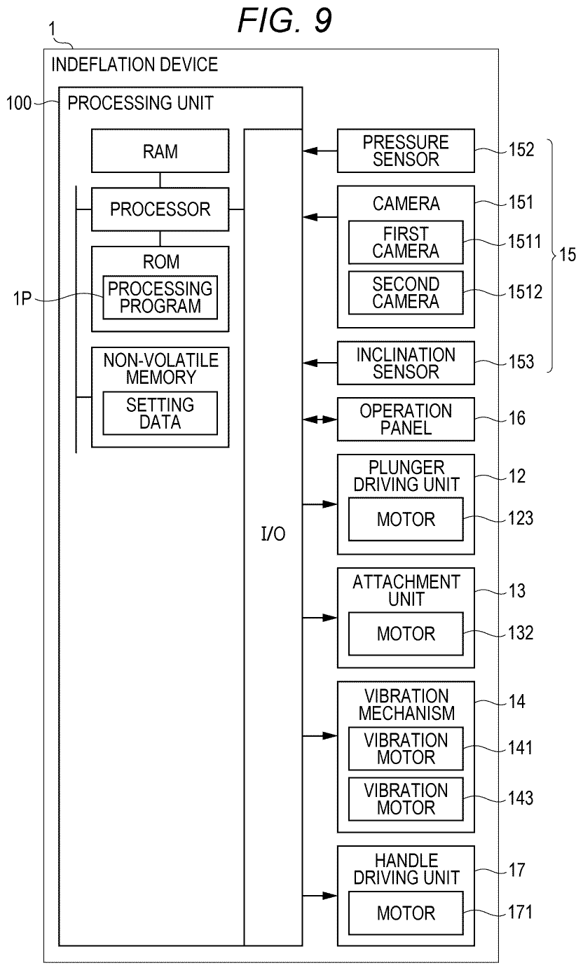

In a second embodiment, a valve to an air reservoir or an exhaust port, in a tube 21 connected to a catheter 3 of an indeflator 2. FIG. 8 is a schematic perspective view of the indeflator 2 and an indeflation device 1 according to the second embodiment, and FIG. 9 is a block diagram illustrating a configuration of the indeflation device 1 according to the second embodiment. Since a configuration of a medical system 200 according to the second embodiment is similar to the configuration of the medical system 200 according to the first embodiment, except for a part of the configuration of the indeflator 2 and the indeflation device 1 and details of processing according to that, a common component is denoted with the same reference numeral, and detailed description of the configuration of the indeflator 2 and the indeflation device 1, and details of the processing according to that is omitted.

As illustrated in FIG. 8, in the indeflator 2 used for the medical system 200 according to the second embodiment, an air reservoir 211 is provided via a valve 212 at a connection portion between the tube 21 and the syringe 22. The valve 212 can be, for example, a T shape stopcock and includes a T-shaped path therein, and can switch a path communicating between the syringe 22, the tube 21, and the air reservoir 211, between two states. A first state is a state where an inside of the tube 21 communicates with the syringe 22 and the air reservoir 211 communicates with none of them. A second state is a state where the syringe 22 communicates with an inside of the air reservoir 211 and the tube 21 communicates with none of them. In the valve 212, a handle for switching the first state and the second state is provided. Instead of the air reservoir 211, one that includes an opening and functions as an exhaust port may be provided via the valve 212.

The indeflation device 1 according to the second embodiment includes a handle driving unit 17 that automatically operates the handle described above of the valve 212, in correspondence with the indeflator 2 in which the air reservoir 211 is provided in the tube 21. The handle driving unit 17 includes a motor 171 and controls opening/closing of the valve 212 by operating the handle in response to an instruction from a processing unit 100.

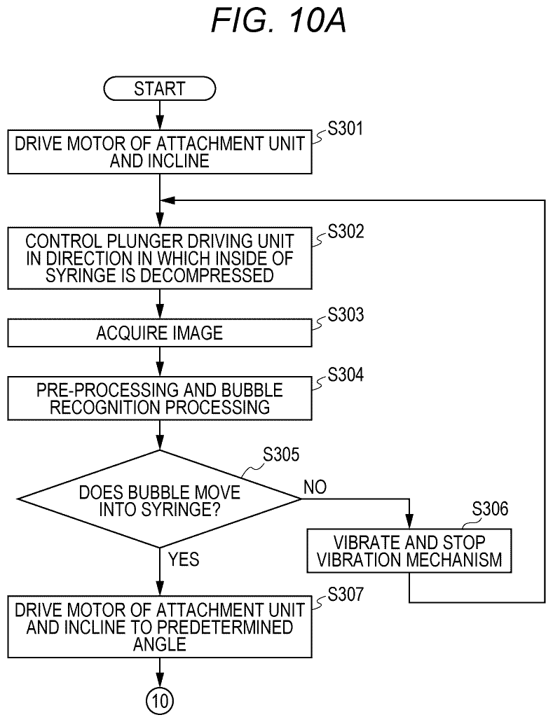

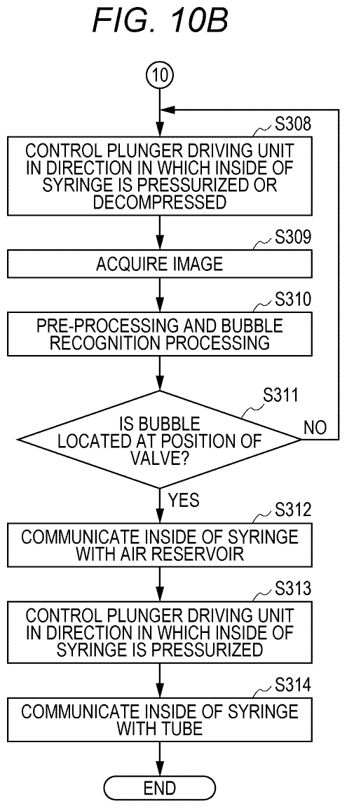

FIGS. 10A and 10B are flowcharts illustrating an example of a processing procedure by the processing unit 100 according to the second embodiment. The processing procedure illustrated in the flowcharts in FIGS. 10A and 10B is executed in a case where it is determined that the bubble exists in the tube 21 in S104 in the processing procedure illustrated in the flowcharts in FIGS. 5 to 7 in the first embodiment. In a case where the indeflator 2 in which the valve 212 connected to the air reservoir 211 is a target in the processing procedure illustrated in the flowcharts in FIGS. 5 to 7, the processing unit 100 executes the following processing, instead of the processing in S105 to S114.

The processing unit 100 inclines the indeflation device 1 at a predetermined angle by driving a motor 132 of an attachment unit 13 (S301).

In order to move the bubble in the syringe 22, the processing unit 100 controls a plunger driving unit 12 so as to move a plunger 23 in a direction in which an inside of the syringe 22 is decompressed for a predetermined time (for example, five seconds) (S302). After a predetermined time has elapsed, the processing unit 100 moves the plunger 23 to a position before the decompression.

The processing unit 100 acquires an image after the predetermined time has elapsed (S303), executes pre-processing and recognition processing on the acquired image (S304), and determines whether or not the bubble in the tube 21 has moved to the syringe 22 or a nozzle of the syringe 22 (S305).

In a case where it is determined that the bubble does not move to the syringe 22 or the nozzle of the syringe 22 (S305: NO), the processing unit 100 vibrates a vibration motor 141 at a position closest to the nozzle of the syringe 22, for a predetermined time (for example, three seconds) (S306) and returns the processing to S302. In S306, the processing unit 100 may vibrate the vibration motor 143 by moving the slider 142 to the position closest to the nozzle of the syringe 22. The processing unit 100 may further increase an inclination angle in S306 or may add vibration.

In a case where it is determined that the bubble has moved to the syringe 22 or the nozzle of the syringe 22 (S305: YES), the processing unit 100 sets an inclination of the indeflation device 1, that is, the indeflator 2 to a predetermined inclination angle (S307). Here, the predetermined inclination angle is an angle set in advance so as not to move the bubble from the valve 212 to the side of the catheter 3 of the tube 21, while moving the bubble to the position of the valve 212, as described later.

In order to move the detected bubble to the position of the valve 212, the processing unit 100 controls the plunger driving unit 12 so as to move the plunger 23 in a pressurizing or decompressing direction (S308).

The processing unit 100 acquires an image from a camera 151 (S309), executes the pre-processing and the recognition processing on the acquired image (S310), and determines whether or not the bubble has moved to the position of the valve 212 (S311). In S311, the processing unit 100 may determine whether or not the bubble cannot be confirmed (in valve 212), in an image from a first camera 1511 and an image from a second camera 1512.

In a case where it is determined that the bubble does not move to the position of the valve 212 (S311: NO), the processing unit 100 returns the processing to S308 and moves the bubble by driving the plunger driving unit 12.

In a case where it is determined that the bubble has moved to the position of the valve 212 (S311: YES), the processing unit 100 controls the handle driving unit 17 so that the handle of the valve 212 is in a direction in which the inside of the syringe 22 communicates with the air reservoir 211 (S312).

The processing unit 100 controls the plunger driving unit 12 so as to move the plunger 23 in a pressurizing direction, in order to remove the bubble (S313). At this time, the movement of the plunger driving unit 12 to remove the bubble is controlled to stop when a pressure obtained by a pressure sensor 152 reaches a pressure value at which the pressure presses and moves liquid. This is because, since the bubble has a higher compressibility than liquid, the pressure value when the bubble is moved is smaller than the pressure value when the liquid is moved. At this time, regarding the movement of the plunger driving unit 12 in order to remove the bubble, a movement amount set in advance so as to move a volume of liquid from the valve 212 to the air reservoir 211 is set as a lower limit. Furthermore, a movement amount that causes liquid having a volume larger than preset value to remain in the syringe 22 is set as an upper limit of the movement of the plunger driving unit 12.

When the pressure reaches the pressure value for pressing the liquid, the processing unit 100 controls the handle driving unit 17 so that the handle of the valve 212 is in a direction in which the inside of the syringe 22 communicates with the tube 21 (S314) and ends the processing.

In S313, the processing unit 100 may acquire the image from the camera 151, execute the bubble recognition processing, and repeat the control for pressurizing the inside of the syringe 22 until the removal of the bubble can be confirmed. In this case, only after the removal of the bubble is confirmed according to the image obtained from the camera 151, the processing unit 100 stops the movement of the plunger driving unit 12 and causes the inside of the syringe 22 to communicate with the tube 21 by the valve 212 (S314), and ends the processing.

The control for performing the pressurization by the plunger driving unit 12 in order to remove the bubble to the air reservoir 211 (or exhaust port) in the second embodiment may be similarly performed in a case where the bubbles exist only in the syringe 22. In order to remove the bubbles, the control of the inclination and the vibration may be combined.

### Third Embodiment

The indeflation device 1 according to the first embodiment and the second embodiment includes the sensor group 15 and executes the image processing on the image acquired from the camera 151, the analysis processing of bubble detection, and the processing for determining the control content. However, the present invention is not limited to this, and the indeflation device 1 may perform inclination, vibration, or the like in response to an instruction based on determined control content, as a result of executing the analysis processing by an external device and the processing for determining the control content.

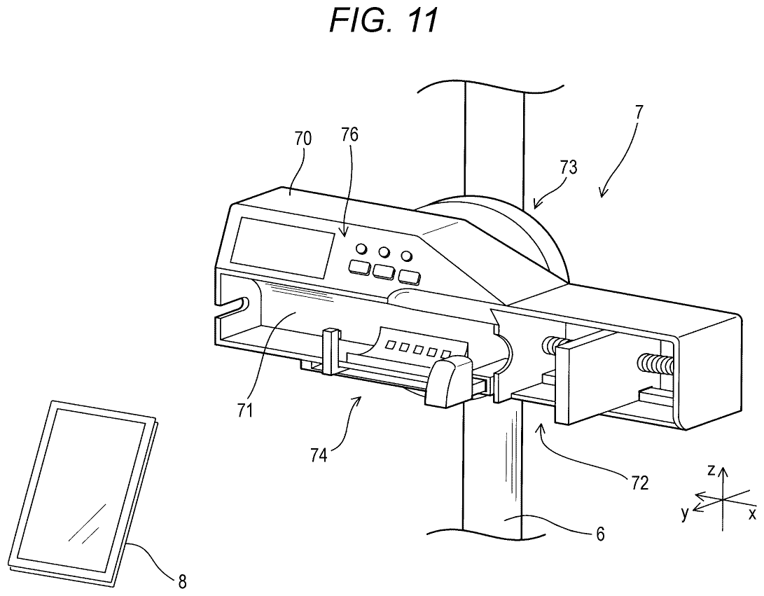

FIG. 11 is a schematic view of an indeflation system 300 according to a third embodiment. The indeflation system 300 includes an indeflation device 7 and a control device 8. The indeflation device 7 according to the third embodiment has a hardware configuration similar to the indeflation device 1 according to the first embodiment, except that the indeflation device 7 does not include a camera and includes a communication unit 77. The indeflation device 7 includes a processing unit 700, a pressure sensor 752, and an inclination sensor 753 in a casing 70. The indeflation device 7 includes a holding portion 71, a plunger driving unit 72, an attachment unit 73, a vibration mechanism 74, and an operation panel 76 in the casing 70. Since components other than processing content by the processing unit 700 are similar to the pressure sensor 152, the inclination sensor 153, the holding portion 11, the plunger driving unit 12, the attachment unit 13, the vibration mechanism 14, and the operation panel 16 of the indeflation device 1 according to the first embodiment, corresponding reference numerals are applied, and detailed description is omitted.

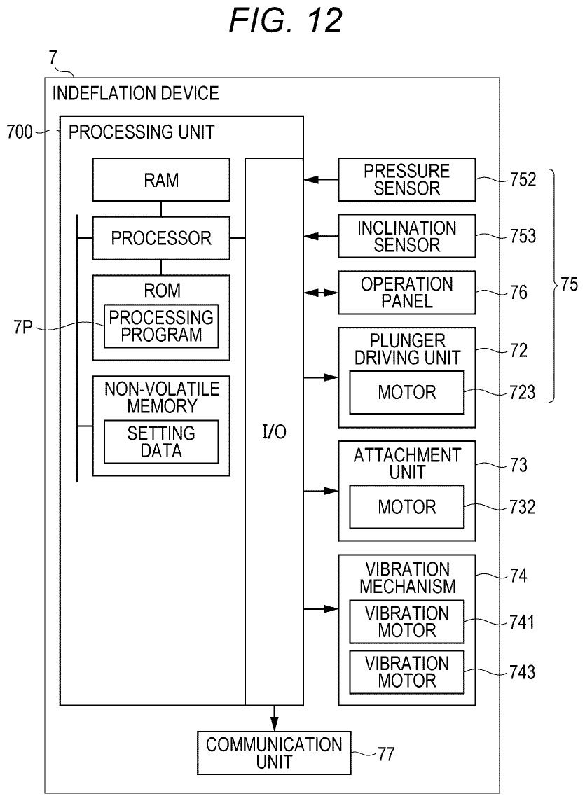

FIG. 12 is a block diagram illustrating a configuration of the indeflation device 7 according to the third embodiment. The indeflation device 7 according to the third embodiment includes the communication unit 77. The communication unit 77 can be a wireless communication module that realizes near field communication. The communication unit 77 enables the indeflation device 7 to communicate with the control device 8. The communication unit 77 may be a wired communication module such as a universal serial bus (USB), not limited to the near field communication.

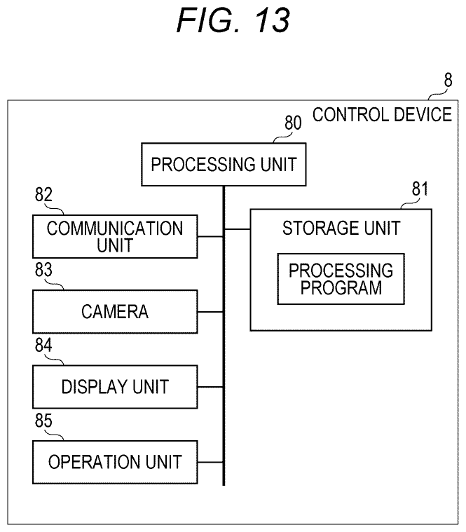

FIG. 13 is a block diagram illustrating a configuration of the control device 8. The control device 8 can be a portable communication terminal such as a smartphone or a tablet terminal owned by a user. The control device 8 includes a processing unit 80, a storage unit 81, a communication unit 82, a camera 83, a display unit 84, and an operation unit 85.

The processing unit 80 can be, for example, a CPU, an MPU, a graphics processing unit (GPU), general-purpose computing on graphics processing units (GPGPU), a tensor processing unit (TPU), or the like. The processing unit 80 creates control data indicating the control content of the indeflation device 7 as described later, by reading and executing a processing program 8P stored in the storage unit 81.

The storage unit 81 is a non-volatile storage medium such as a hard disk or a flash memory. The storage unit 81 stores the processing program 8P read by the processing unit 80, setting data, or the like. The setting data is data for bubble detection and data of a first size, a second size, or the like.

The communication unit 82 is a wireless communication module for communicating with the indeflation device 7. The communication unit 82 may be wired or wireless as long as the communication unit 82 is a module that can realize communication according to a communication standard corresponding to the communication unit 77 of the indeflation device 7.

The camera 83 is a module that includes a lens provided outward in a casing of the control device 8 and can image a picture or a moving image by a user of the control device 8. When being activated, the camera 83 monitor-outputs an image signal of an object existing in an angle of view, and the processing unit 80 can sequentially capture (acquire) the image from the image signal from the camera 83.

The display unit 84 is a display such as a liquid crystal display or an organic electro luminescence (EL) display. The display unit 84 is a display with a built-in touch panel, for example. The processing unit 80 displays an operation screen including a monitor output screen from the camera 83, on the display unit 84, on the basis of the processing program 8P.

The operation unit 85 can be, for example, a touch panel built in the display unit 84. The operation unit 85 may be a physical button. The operation unit 85 may be a sound input unit.

In the indeflation system 300 configured in this way, a risk that the bubbles existing in the syringe 22 of the indeflator 2 or the tube 21 move from the tube 21 to the distal end can be reduced. An operator houses the holding portion 71 of the indeflation device 7 in the indeflator 2 filled with a fluid such as a contrast agent or a normal saline solution, fixes the indeflator 2 with a clamp, activates the processing program 8P of the control device 8, and positions the camera 83 with respect to the syringe 22 and the tube 21 on the basis of the monitor output from the camera 83 included in the operation screen. When the operator activates the indeflation device 7, the indeflation system 300 starts the following processing.

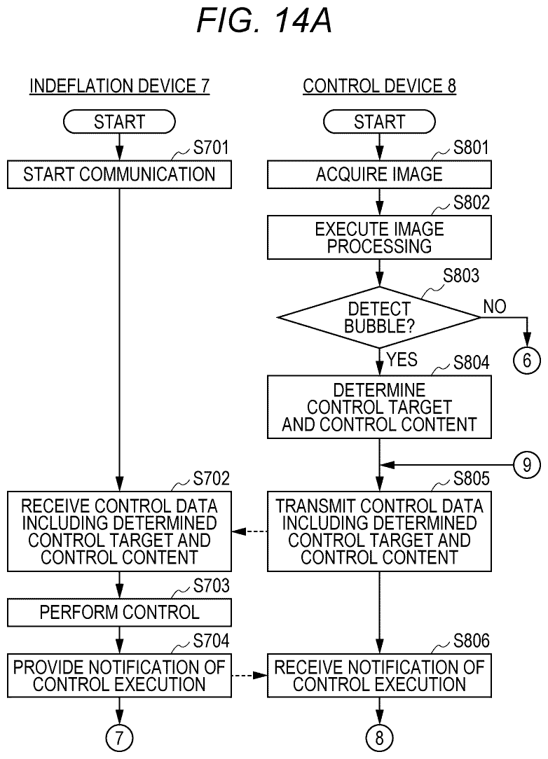

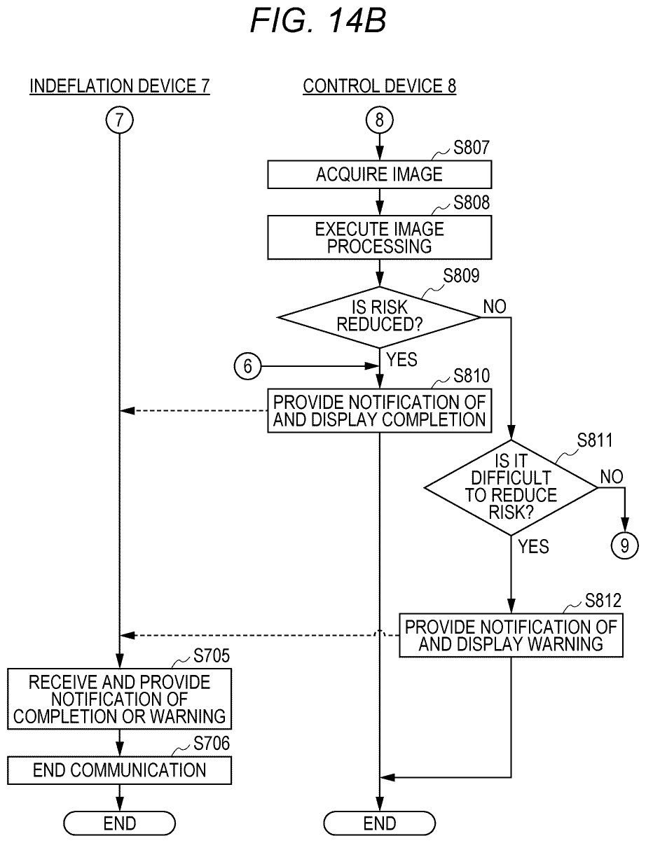

FIGS. 14A and 14B are flowcharts illustrating an example of a processing procedure of the indeflation system 300 according to the third embodiment.

The processing unit 700 of the indeflation device 7 starts communication with the control device 8 (S701) and stands by.

The processing unit 80 of the control device 8 acquires the image from the camera 83 (S801) and executes image processing for detecting whether or not the bubble exists and a size and a position of the bubble, on the acquired image (S802). Details of the image processing is similar to the processing procedure illustrated in the flowcharts in FIGS. 5 to 7 in the first embodiment.

The processing unit 80 determines whether or not the bubble is detected in the tube 21 or the syringe 22, as a result of the image processing (S803).

In a case where it is determined that the bubble is detected (S803: YES), the processing unit 80 executes processing for determining one or a plurality of control targets among the attachment unit 73 that is an inclination mechanism of the indeflation device 7, the vibration mechanism 74, and the plunger driving unit 72 and control content associated with the attachment unit 73, the vibration mechanism 74, and the plunger driving unit 72, on the basis of the size or the position of the bubble (S804). The determination method in S804 is similar to the processing procedure illustrated in the flowcharts in FIGS. 5 to 7 in the first embodiment.

The processing unit 80 transmits control data including the control target and the control content determined in S804 to the indeflation device 7 (S805).

When the indeflation device 7 during standby receives the control data including the control target and the control content (S702), the processing unit 700 performs control for operating one, two, or all of a motor 732 of the attachment unit 73, vibration motors 741 and 743 of the vibration mechanism 74, and a motor 723 of the plunger driving unit 72 on the basis of the control data (S703). The processing unit 700 provides a notification of control execution (S704).

When receiving a notification of the control execution (S806), the processing unit 80 of the control device 8 acquires an image again from the camera 83 that images the indeflator 2 (S807) and executes the image processing for detecting whether or not the bubble exists and the size and the position of the bubble (S808).

As a result of the image processing on the image acquired in S807, it is determined whether or not a risk that the bubble detected in S803 moves from the tube 21 to the catheter 3 is sufficiently reduced (S809). Since the determination processing in S809 is similar to the processing content in S119 in the first embodiment, detailed description of the determination processing in S809 is omitted.

In a case where it is determined that the risk is sufficiently reduced (S809: YES), the processing unit 80 displays control completion on the operation screen while providing the indeflation device 7 with a notification that the control by the indeflation device 7 has been completed (S810) and ends the processing.

In a case where it is determined in S809 that the risk is not sufficiently reduced (S809: NO), the processing unit 80 determines whether or not it is difficult to reduce the risk (S811). In S811, it is determined that the risk reduction is difficult, for example, in a case where the bubble does not move although the processing unit 80 transmits the control data a predetermined number of times of executions or more.

In a case where it is determined that the risk reduction is difficult (S811: YES), the processing unit 80 displays a warning on the operation screen while providing the indeflation device 7 with a notification of the warning (S812) and ends the processing.

In a case where it is determined that the risk reduction is not difficult (S811: NO), the processing unit 80 returns the processing to S805 and continues control according to the similar control content. In this case, the processing unit 80 may execute the processing from S801 again.

The indeflation device 7 receives the notification of the control completion, provides a notification of the control completion from the operation panel 76 in response to this (S705), ends the communication by the communication unit 77 (S706), and ends the processing.

In a case where it is determined in S803 that the bubble is not detected (S803: NO), the processing unit 80 of the control device 8 proceeds the processing to S810. In this case, the processing unit 700 of the indeflation device 7 receives the notification of the control completion and provides a notification of the notification content from the operation panel 76 (S705), ends the communication (S706), and ends the processing.

In this way, the indeflation device 7 leaves the determination based on the image processing regarding whether or not the bubble exists and the control content determination processing to an external calculation resource. By using the indeflation device 7 including the inclination mechanism (attachment unit 73) that inclines the indeflator 2, the vibration mechanism 74 that vibrates the indeflator 2, and the plunger driving unit 72 that decompresses the inside of the syringe 22, it is possible to avoid the risk that the bubble is mixed from the indeflator 2 into the side of the catheter 3.

### Fourth Embodiment

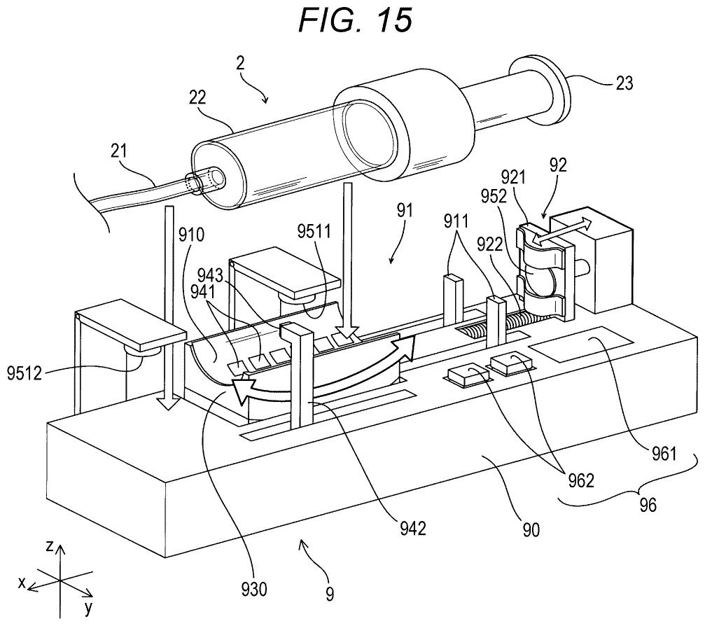

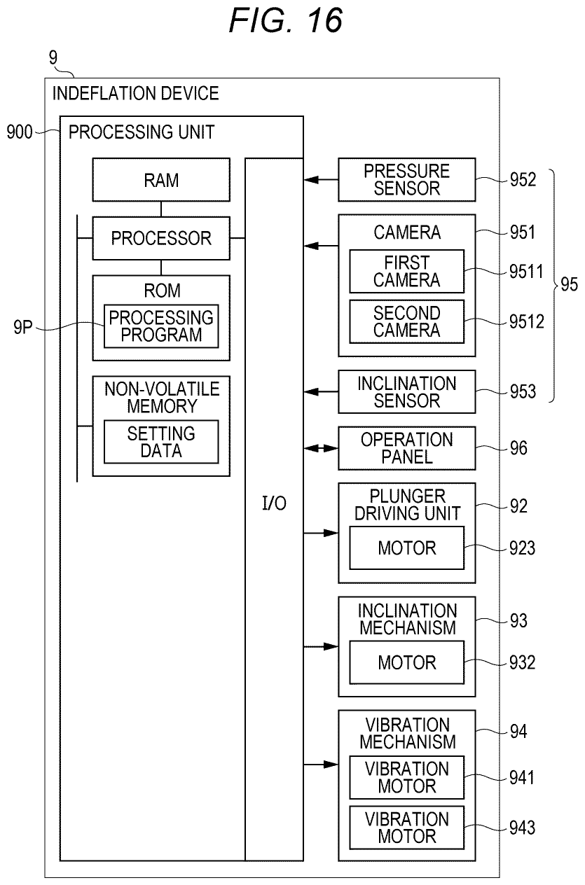

FIG. 15 is a schematic perspective view of an indeflation device 9 according to a fourth embodiment, and FIG. 16 is a block diagram illustrating a configuration of the indeflation device 9 according to the fourth embodiment. As illustrated in FIG. 15, the indeflation device 9 according to the fourth embodiment is not attached to a support column and is used to be installed on a desk.

The indeflation device 9 according to the fourth embodiment includes a casing 90, a holding portion 91 that holds an indeflator 2, a plunger driving unit 92 that moves a plunger 23 of a syringe 22 of the indeflator 2, an inclination mechanism 93 that inclines the indeflator 2, and a vibration mechanism 94 that vibrates the indeflator 2. The indeflation device 9 includes a sensor group 95 that specifies a state of the indeflator 2, an operation panel 96, and a processing unit 900 that executes processing for controlling each unit.

The holding portion 91 has a curved holding surface 910 along a part of an outer cylinder side surface of the syringe 22 of the indeflator 2. At one end of the holding surface 910 in a length direction, a hook used to fix the length direction of the syringe 22 is erected perpendicularly to the holding surface 910. Another end of the holding surface 910 in the length direction is released so as to be adapted to various indeflators 2. A pressing tool 911 that presses and holds the syringe 22 is provided on the side of the plunger 23 so as to be slidable in a length direction of the casing 90, and the length direction of the indeflator 2 is fixed by the pressing tool 911.

The plunger driving unit 92 includes a slider 921 having a surface in contact with an end surface of the plunger 23 of the indeflator 2 held by the holding portion 91. The slider 921 is bidirectionally movable by a feed screw that rotates by driving of a motor provided in the casing 90.

As illustrated in FIG. 15, the inclination mechanism 93 is configured such that the holding surface 910 is attached to a stage rotatable about an axis perpendicular to the length direction of the holding surface 910, with respect to a base portion fixed to the casing 90. By a mechanism in which a wheel and a worm screw are connected to a rotation shaft of the stage and the worm screw is rotated by the motor, it is possible to automatically adjust an inclination. Note that the rotation axis of the inclination is not limited to the above.

The vibration mechanism 94 includes a plurality of vibration motors 941 provided along the length direction, on the holding surface 910 of the holding portion 91. The vibration mechanism 94 includes a vibration motor 943 that is attached to a slider 942 movable in the length direction with respect to the casing 90 and has contact with the outer cylinder side surface of the syringe 22. It is sufficient for the vibration mechanism 94 to include at least one of the vibration motor 941 provided on the holding surface 910 and the vibration motor 943 attached to the slider 942.

The sensor group 95 includes a camera 951 of which an imaging range is the syringe 22 and the tube 21, a pressure sensor 952 that estimates a pressure in the syringe 22, and an inclination sensor 953 that detects an inclination of the indeflator 2, that is, the syringe 22.

The camera 951 includes a first camera 9511 that images the syringe 22 and a second camera 9512 that images the tube 21. The first camera 9511 and the second camera 9512 may further include a plurality of cameras.

The first camera 9511 is fixed to the casing 90 above the holding surface 910 at an angle of view at which an inside of a transparent or translucent outer cylinder of the syringe 22 can be imaged. The first camera 9511 may image the inside of the outer cylinder of the syringe 22 from the side and from the side of the holding portion 91 or from both of the side and an upper side.

The second camera 9512 is fixed to the casing 90 at an angle of view at which an inner surface of the transparent or translucent tube 21 extending from a nozzle of the syringe 22 held by the holding portion 91 can be imaged. The second camera 9512 monitor-outputs an image signal of an image capturing the inner surface of the tube 21 being imaged in the angle of view. The second camera 9512 may image the inner surface of the tube 21 from the side or from both of the side and the upper side.

The pressure sensor 952 is provided on a contact surface of the plunger 23 with the slider 921 of the plunger driving unit 92. The pressure sensor 952 measures a reaction force from the plunger 23 in a case where the slider 921 is moved to pressurize the syringe 22, as a pressure. As the pressure sensor 952, a load cell, a strain sensor, or the like is used. The pressure sensor 952 outputs a signal corresponding to the measured reaction force to the processing unit 900. The pressure sensor 952 may also measure a pressure in the syringe 22.

The inclination sensor 953 is provided in a part of the holding surface 910 inclined by the inclination mechanism 93. The inclination sensor 953 detects an angle from the horizontal direction and outputs the angle to the processing unit 900. The processing unit 900 can detect an inclination of the held indeflator 2 from the horizontal direction, according to the output from the inclination sensor 953.

The operation panel 96 includes a display 961 and a physical button 962 that receives an operation. The physical buttons 962 include a start button and a stop button. Note that the operation panel 96 may include not only the physical buttons 962 but also a touch panel built in the display 961 and may receive an operation with the touch panel, instead of the physical button 962.

The processing unit 900 is fixed in the casing 90, and is connected to the plunger driving unit 92, a motor of the inclination mechanism 93, the vibration motors 941 and 943 of the vibration mechanism 94, the sensor group 95, and the operation panel 96 with signal lines.

The processing unit 900 includes a processor such as a CPU or an MPU and a memory such as a ROM or a RAM. The processing unit 900 can be, for example, a microcontroller. The ROM stores a processing program 9P, setting data, or the like. The processor takes the image signal obtained from the camera 951, in response to the operation received by the operation panel 96, on the basis of this processing program 9P, and executes control processing of any one or the plurality of the plunger driving unit 92, the inclination mechanism 93, and the vibration mechanism 94, on the basis of the image. The setting data is data for bubble detection used in processing to be described later and data of a first size, a second size, or the like.

In the indeflation device 9 according to the fourth embodiment, in order to move the bubble toward the plunger 23 in a case where the bubble exists, as in the first embodiment, the indeflator 2 held by the holding portion 91 is configured to be inclined by the inclination mechanism 93, even in a mode illustrated in FIG. 15, and the indeflator 2 can be inclined so as to raise the side of the plunger 23 of the indeflator 2. Furthermore, the indeflation device 9 is configured to be able to vibrate the vibration mechanism 94 to apply stimulation to the bubbles in the fluid. Moreover, the indeflation device 9 is configured to be able to draw the plunger 23 by the plunger driving unit 92 in order to move the bubbles and draw the bubbles from the tube 21 into the syringe 22.

The processing unit 900 of the indeflation device 9 according to the fourth embodiment can perform the processing procedure similar to that in the first embodiment. Therefore, detailed description of the processing procedure is omitted.

As described in the first to fourth embodiments, the indeflation devices 1, 7, and 9 respectively include the inclination mechanisms (attachment unit) 13, 73, and 93 and the vibration mechanisms 14, 74, and 94 and can avoid the risk that the bubble existing in the indeflator 2 is mixed into the catheter 3.

The detailed description above describes to an indeflation device and an indeflation system for retracting a bubble from an indeflator used to a medical catheter with liquid and an indeflation system. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An indeflation device comprising:
a holding portion configured to hold an indeflator connected to a medical catheter;
a plunger driving unit configured to move a plunger of a syringe of the indeflator held by the holding portion;
a mechanism configured to incline or vibrate the holding portion; and
a control unit configured to control driving of at least one of the plunger driving unit and the mechanism.

2. The indeflation device according to claim 1, wherein
the mechanism configured to incline or vibrate the holding portion includes an attachment unit configured to incline the holding portion;
a casing in which the holding portion is provided; and
wherein the attachment unit is rotatably provided along one surface of the casing substantially parallel to a length direction, about an axis orthogonal to the length direction of the indeflator held by the holding portion with respect to the casing, and in which a fixing tool to a support that supports the casing is provided so as to maintain the one surface to be in a vertical direction.

3. The indeflation device according to claim 1, wherein
the mechanism configured to incline or vibrate the holding portion includes an attachment unit configured to incline the holding portion; and
a casing in which a stage that rotates along a substantially vertical surface, about an axis orthogonal to the length direction of the indeflator held by the holding portion.

4. The indeflation device according to claim 2, further comprising:
an inclination sensor configured to measure an inclination from a horizontal surface of the holding portion, the inclination sensor being provided in the attachment unit; and
the control unit is configured to control the attachment unit on a basis of the inclination measured by the inclination sensor.

5. The indeflation device according to claim 1, wherein
the control unit is configured to control one or more of the plunger driving unit and an inclination or a vibration of the mechanism on a basis of data regarding a bubble in the indeflator held by the holding portion.

6. The indeflation device according to claim 1, further comprising:
a camera configured to image the indeflator held by the holding portion; and
wherein the control unit is configured to control one or more of the plunger driving unit and the inclination or the vibration of the mechanism, on a basis of at least one of whether or not a bubble captured in an image imaged by the camera exists, a position of the bubble, a size of the bubble, and the number of bubbles.

7. The indeflation device according to claim 6, wherein
the control unit is configured to control one or more of the plunger driving unit and the inclination or the vibration of the mechanism, so as to move the bubble to a desired position in the indeflator, on a basis of the image obtained from the camera.

8. The indeflation device according to claim 7, wherein
the desired position is a position of a valve to an air reservoir or an exhaust port provided in the indeflator.

9. The indeflation device according to claim 7, wherein
the control unit is configured to control the plunger driving unit so as to move the plunger in a direction of decompressing or pressurizing an inside of the syringe of the indeflator.

10. The indeflation device according to claim 1, wherein
the mechanism configured to incline or vibrate the holding portion includes a vibration mechanism configured to vibrate the indeflator held by the holding portion in the mechanism is a vibration motor that has contact with the indeflator at any position in the length direction of the indeflator.

11. The indeflation device according to claim 10, wherein
the vibration mechanism includes a plurality of vibration motors provided along the length direction of the indeflator of the holding portion.

12. The indeflation device according to claim 10, wherein
the vibration mechanism includes a slider that moves along the length direction of the indeflator of the holding portion and a vibration motor provided in the slider.

13. The indeflation device according to claim 10, wherein
the control unit is configured to determine a position where the vibration motor is vibrated, on a basis of the position of the bubble imaged by a camera configured to image the indeflator held by the holding portion.

14. The indeflation device according to claim 13, wherein
the control unit is configured to determine an intensity of vibration by the vibration motor, on a basis of a change amount of the position of the bubble imaged by the camera.

15. An indeflation system comprising:
an indeflation device including a holding portion configured to hold an indeflator connected to a medical catheter, a plunger driving unit configured to move a plunger of a syringe of the indeflator held by the holding portion, and a mechanism configured to incline or vibrate the holding portion; and
a control device configured to determine which one of the plunger driving unit and an inclination or a vibration of the mechanism is driven and to instruct the indeflation device on determined control content.

16. A method for retracting a bubble from an indeflation system, the method comprising:
holding an indeflator connected to a medical catheter in a recess of a casing of the indeflation system; and
one or more of moving a plunger of a syringe of the indeflator, the syringe of the indeflator being held in the recess of the inflation system, and inclining or vibrating the recess of the casing of the indeflation system.

17. The method according to claim 16, further comprising:
measuring an inclination from a horizontal surface of the recess of the casing with an inclination sensor; and
controlling the inclination of the recess of the casing on a basis of the inclination measured by the inclination sensor.

18. The method according to claim 16, further comprising:
controlling one or more of the movement of the plunger and the inclination or the vibration on a basis of data regarding a bubble in the indeflator held in the recess of the casing of the indeflator system.

19. The method according to claim 16, further comprising:
imaging the indeflator held in the recess of the casing of the indeflator system; and
controlling one or more of the movement of the plunger and the inclination or the vibration of the recess of the casing of the indeflator system, on a basis of at least one of whether or not a bubble captured in the imaging of the indeflator, a position of the bubble, a size of the bubble, and the number of bubbles.

20. The method according to claim 19, further comprising:
controlling one or more of the movement of the plunger and the inclination or the vibration of the recess of the casing of the indeflator system, so as to move the bubble to a desired position in the indeflator, on a basis of the image of the indeflator held in the recess of the casing the indeflator system.
